(19) United States
(12) Patent Application Publication
(10) Pub. No.: US 2014/0121193 A1
(43) Pub. Date: May 1, 2014

(54) METHODS FOR TREATING FIBROMYALGIA

(71) Applicants: Robert S. KATZ, Chicago, IL (US); Frank LEAVITT, Chicago, IL (US)

(72) Inventors: Robert S. KATZ, Chicago, IL (US); Frank LEAVITT, Chicago, IL (US)

(21) Appl. No.: 14/070,047

(22) Filed: Nov. 1, 2013

Related U.S. Application Data
(60) Provisional application No. 61/721,466, filed on Nov. 1, 2012.
(60) Provisional application No. 61/724,819, filed on Nov. 9, 2012.

(51) Int. Cl. A61K 31/4458; A61K 45/06; A61K 31/155; A61K 31/137; A61K 31/138

(52) U.S. Cl. CPC A61K 31/4458; A61K 31/137; A61K 31/138; A61K 31/155; A61K 45/06
USPC 514/210.16; 514/317; 514/319; 514/654; 514/647; 514/651; 514/634; 514/259.41; 514/235.8; 514/231.5; 514/253.07

(57) ABSTRACT

The invention provides methods for treating or ameliorating cognitive dysfunction, fatigue, energy, concentration, mood, and pain associated with fibromyalgia using compositions containing methylphenidate or pharmaceutically equivalents thereof.

This application claims the benefit under 35 U.S.C. §119(e) of U.S. Provisional Application No. 61/721,466, filed Nov. 1, 2012, and U.S. Provisional Application No. 61/724,819, filed Nov. 9, 2012, the disclosures of which are incorporated herein by reference in their entirety.

### FIELD OF THE INVENTION

The invention provides methods for treating or ameliorating cognitive dysfunction, fatigue, and pain associated with fibromyalgia using compositions containing methylphenidate.

### BACKGROUND OF THE INVENTION

Fibromyalgia is chronic widespread pain associated with insomnia, fatigue, and cognitive dysfunction. Surveys in the United States estimate that fibromyalgia affects approximately 2-5% of the adult population (Lawrence et al., 2008, “Estimates of the prevalence of arthritis and other rheumatic conditions in the United States. Part II.” Arthritis Rheum. 58(1): 26-35). Currently, there is no objective test for fibromyalgia, but it has become a much more respectable diagnosis since the American College of Rheumatology (ACR) defined the illness in 1990 using agreed-upon criteria. However, approximately 25% of fibromyalgia patients do not satisfy the ACR 1990 classification criteria (Wolfe et al., 2010, Arthritis Care Res. 62(5): 600-10). Fibromyalgia may qualify for a significant percentage of the 10-12% of the U.S. population that suffers from chronic pain. In 2010, fibromyalgia criteria were changed, primarily because the requirement of having 11 tender points over a possible 18 throughout the body was difficult to apply in practice. Also, these tender points vary from time to time and could not be relied on for diagnosis, but the other criteria of widespread pain (up to 19 areas throughout the body), poor sleep, fatigue, and cognitive symptoms, especially memory loss and concentration problems, were maintained as essential criteria for the diagnosis.

While there are guidelines for the diagnosis, and clinicians can agree about a diagnosis, fibromyalgia has no specific test for the illness. Although fibromyalgia does not have a specific treatment or treatment paradigm, three medications have been approved by the FDA for the treatment of fibromyalgia. These are pregabalin (Lyrica®), duloxetine (Cymbalta®), and milnacipran (Savella®). However, recent research indicates that current treatments are really not effective in the reduction of pain or improvement in function in patients with fibromyalgia, and there is still a lack of effective drugs for the treatment of fibromyalgia over time (Wolfe et al., 2012, “Longitudinal Patterns of Analgesic and Central Acting Drug Use and Associated Effectiveness in Fibromyalgia,” Eur. J. Pain 17(4): 581-86). With the lack of effective drugs for the treatment of fibromyalgia over time, there is a need to look for new effective treatments for fibromyalgia.

### SUMMARY OF THE INVENTION

The invention provides methods for treating or ameliorating cognitive dysfunction, fatigue, energy, concentration, mood, and pain associated with fibromyalgia using compositions containing methylphenidate.

The invention also provides methods for monitoring a patient's progress while being treated using compositions containing methylphenidate.

Specific embodiments of the invention will become evident from the following more detailed description of certain embodiments and the claims.

### DETAILED DESCRIPTION OF THE INVENTION

Given the high percent of fibromyalgia patients that suffer from cognitive impairment, fatigue, and pain, as well as the lack of effective drugs for the treatment of fibromyalgia over time, there is a need to look for new effective treatments for fibromyalgia. The inventors have tested methylphenidate (e.g., Ritalin®, Concerta®, Metadate®, and Methylin®) for efficacy in treating the symptoms of fibromyalgia. The inventors have done much research on the neurocognitive symptoms of fibromyalgia, and have disclosed four reproducible abnormalities in patients with fibromyalgia, and none of these patients evidence dementia as a reason for their poor cognitive function. These patients talk about getting confused, having poor word retrieval skills, and generally getting mixed up easily (Leavitt & Katz, 2008, J. Clin. Rheumatol. 14(4): 214-18; Katz et al., 2004, J. Clin. Rheumatol. 10(2): 53-58; Leavitt et al., 2002, J. Clin. Rheumatol. 8(2): 77-84). The inventors have discovered that fibromyalgia patients have slower processing speeds for certain types of testing, and have difficulty with the Auditory Consonant Trigram test, a measure of one's ability to recall rote verbal information over a distractor.

On the basis of these neurocognitive findings, the inventors have determined that fibromyalgia patients have problems of distraction interfering with their thinking and comprehension as well as slowness in certain recall processes. Because attention deficit disorder is similar in that distraction prevents people with this problem from focusing their attention, the inventors have applied a similar treatment to fibromyalgia. Using a Mental Clutter Scale developed by the inventors, which lists the neurocognitive symptoms of fibromyalgia and provides a way of following cognitive complaints, the inventors observed significant cognitive improvement in fibromyalgia patients treated with methylphenidate. Surprisingly, the inventors have seen that many patients report significant improvement in all their symptoms, including cognitive dysfunction, fatigue, and pain, with the use of methylphenidate.

### 1. GENERAL DEFINITIONS

As utilized in accordance with the present disclosure, the following terms, unless otherwise indicated, shall be understood to have the following meanings. Unless otherwise required by context, singular terms shall include pluralities and plural terms shall include the singular.

The term “patient” as used herein refers to a human subject.

A “disease” or “disorder” is any condition that would benefit from treatment using the methods or compositions of the invention. “Disease,” “disorder,” and “condition” are used interchangeably herein and include chronic and acute disorders or diseases.

The compositions and methods of the invention can be used to treat or ameliorate symptoms associated with fibromyalgia. In a preferred method, use, or composition of the invention, it can be used to treat or ameliorate cognitive dysfunction, fatigue, or pain associated with fibromyalgia.

The term “treat” as used herein refers to both therapeutic treatment and prophylactic or preventative measures. The term “treatment” as used herein refers to the alleviation of symptoms of a disease. Those in need of treatment include those having the disorder as well as those prone to have the disorder or those in which the disorder is to be prevented.

The terms “composition,” “therapeutic composition,” or “pharmaceutical composition” as used herein refer to a compound or composition capable of inducing a desired therapeutic effect when properly administered to a patient.

The term “therapeutically acceptable” as used herein refer to one or more formulation materials suitable for accomplishing or enhancing the delivery of a composition of the invention.

The term “functional improvement” as used herein refers to a partial and/or complete, but in any case, significant improvement or restoration of at least one aspect of normal function to a state observed before onset of fibromyalgia symptoms. This is determined for each patient, e.g., by comparing the functional improvement following drug treatment with reference to the functional efficiency in a healthy population or before onset of fibromyalgia symptoms. Typically, the determination of improvement in one or more features of fibromyalgia recognized by the American College of Rheumatology criteria for diagnosis (e.g., dyscognition, fatigue, pain, energy, mood, muscle pain, irritable bowel syndrome, thinking or remembering problems, muscle weakness, headache, numbness/tingling, dizziness, insomnia, and depression) will be indicative of overall functional improvement. Most tests combine one or more of these aspects of features of fibromyalgia. Functional restoration or improvement of features of fibromyalgia can for instance be evaluated and/or quantified using the Health Assessment Questionnaire (HAQ), which is a short measurement questionnaire regarding patient function. HAQ scores are calculated based on a patient checking a short form about the activities of daily living that the patient is able to do. Additionally or alternatively, the degree of functional improvement or restoration is measured using a Widespread Pain Index or Symptom Severity scale score. Functional improvement can also prevent disability as many fibromyalgia patients conclude that they can no longer work due to fatigue, cognitive problems, and pain.

The symbol “—” means a single bond, “═” means a double bond and “ [symbol] ” means the bond may be a single bond or a double bond depending on the substituent. For example, if a sp² hybridized carbon atom or heteroatom requiring a double bond, such as, for example, ═CH₂ or oxo, is chosen, the bond will be a double bond. If a sp^{a} hybridized carbon atom or heteroatom requiring a single bond, such as, for example, —CH₃ or —OH, is chosen, the bond will be a single bond, and a hydrogen will be added to the carbon to which the substituent is attached to achieve proper valence.

If a group “R” is depicted as “floating” on a ring system, as for example in the formula:

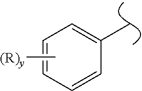

then, unless otherwise defined, a substituent “R” may reside on any atom of the ring system, assuming replacement of a depicted, implied, or expressly defined hydrogen from one of the ring atoms, so long as a stable structure is formed. The number of “R” groups residing on the ring system is defined by y. For example, when y is 2, then the two “R's” may reside on any two atoms of the ring system, again assuming each replaces a depicted, implied, or expressly defined hydrogen on the ring.

“Alkyl” is intended to include linear, branched, or cyclic hydrocarbon structures and combinations thereof, inclusively. For example, “C₈ alkyl” may refer to an n-octyl, iso-octyl, and the like. Lower alkyl (C₁₋₆ alkyl) refers to alkyl groups of from one to six carbon atoms. Examples of lower alkyl groups include methyl, ethyl, propyl, isopropyl, butyl, s-butyl, t-butyl, isobutyl, pentyl, hexyl and the like. Higher alkyl refers to alkyl groups containing more than eight carbon atoms. Exemplary alkyl groups are those of C₂₀ or below. When an alkyl residue having a specific number of carbons is named, all geometric isomers having that number of carbons are intended to be encompassed; thus, for example, either “butyl” or “C₄ alkyl” is meant to include n-butyl, sec-butyl, isobutyl and t-butyl radicals; and for example, “propyl” or “C₃ alkyl” each include n-propyl and isopropyl. More general descriptions, describing the number of carbons in a particular group as a range, is intended to include all sub-ranges within said range. For example, “C₁₋₈ alkyl” is intended to include “C₁₋₈ alkyl,” “C₁₋₆ alkyl” and “C₃₋₅ alkyl.”

“Cycloalkyl” includes cyclic and multi-cyclic hydrocarbon groups of from three to thirteen carbon atoms. Examples of cycloalkyl groups include cyclopropyl, cyclobutyl, cyclopentyl, norbornyl, adamantly, bicyclo[2.2.1]heptane and the like.

“Acyl” refers to groups of from one to ten carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to six carbons.

“Alkoxy”” refers to the group —O-alkyl, for example, including from one to eight carbon atoms of a straight, branched, cyclic configuration, unsaturated chains, and combinations thereof attached to the parent structure through an oxygen atom. Examples include methoxy, ethoxy, propoxy, isopropoxy and the like. Lower-alkoxy refers to groups containing one to six carbons.

“Aryl” refers to aromatic six- to fourteen-membered carbocyclic ring, for example, benzene, naphthalene, indane, tetralin, fluorene and the like, univalent radicals. As univalent radicals, the aforementioned ring examples are phenyl, naphthyl, indanyl, tetralinyl, and fluorenyl.

“Aryloxy” refers to the group —O-aryl, for example, aromatic six- to fourteen-membered carbocyclic ring attached to the parent structure through an oxygen atom. For example, phenoxy and naphthyloxy.

“Arylalkyl” refers to a residue in which an aryl moiety is attached to a parent structure via an alkylene radical. Examples include benzyl, phenethyl, and the like. Both the aryl, and the corresponding alkylene radical portion of an arylalkyl group may be optionally substituted. “Lower arylalkyl” refers to an arylalkyl where the “alkyl” portion of the group has one to six carbons; this can also be referred to as C₁₋₆ arylalkyl.

“Heterocyclyl” refers to a stable three- to ten-membered ring radical that consists of carbon atoms and from one to five heteroatoms selected from the group consisting of nitrogen, phosphorus, oxygen and sulfur. For purposes of this invention, the heterocyclyl radical may be a monocyclic, bicyclic or tricyclic ring system, which may include fused or bridged ring systems as well as spirocyclic systems; and the nitrogen, phosphorus, carbon or sulfur atoms in the heterocyclyl radical may be optionally oxidized to various oxidation states. In a specific example, the group —S(O)₀₋₂—, refers to —S-(sulfide), —S(O)— (sulfoxide), and —SO₂— (sulfone). For nitrogen containing heterocyclyl groups, the corresponding N-oxide forms are included. Thus, for a compound of the invention having, for example, an N-methyl piperidine ring; the corresponding N-methyl piperidyl-N-oxide is meant to be included as another compound of the invention. Examples of heterocyclyl radicals include, but are not limited to, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolidinyl, imidazolidinyl, imidazolinyl, dihydropyridinyl, tetrahydropyridinyl, pyridinyl, morpholinyl, tetrahydrofuryl and tetrahydropyranyl.

“Halogen” or “halo” refers to fluorine, chlorine, bromine or iodine. “Haloalkyl” and “haloaryl” refer generically to alkyl and aryl radicals that are substituted with one or more halogens, respectively. Thus, “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is within the scope of dihaloaryl.

“Optional” or “optionally” means that the subsequently described event or circumstance may or may not occur, and that the description includes instances where said event or circumstance occurs and instances in which it does not. One of ordinary skill in the art would understand that, with respect to any molecule described as containing one or more optional substituents, that only sterically practical and/or synthetically feasible compounds are meant to be included. “Optionally substituted” refers to all subsequent modifiers in a term, for example in the term “optionally substituted arylalkyl,” optional substitution may occur on both the “alkyl” portion and the “aryl” portion of the molecule; and for example, optionally substituted alkyl includes optionally substituted alkyl groups, which in turn are defined as including optionally substituted alkyl groups, potentially ad infinitum. A list of exemplary optional substitutions is included below in the definition of “substituted.”

“Substituted” alkyl, cycloalkyl, alkoxy, aryl, aryloxy, arylalkyl and heterocyclyl, refer respectively to alkyl, alkoxy, aryl, aryloxy, arylalkyl and heterocyclyl, wherein one or more (for example up to about five, in another example, up to about three) hydrogen atoms are replaced by a substituent independently selected from, but not limited to: alkyl, acyl, alkoxy, haloalkyl, haloalkoxy, aryl, aryloxy, arylalkyl, heterocyclyl, halogen, hydroxy, amino, alkylamino or dialkylamino. The resulting substituted groups include: substituted alkyl (for example, fluoromethyl, hydroxypropyl, nitromethyl, amino ethyl and the like), substituted cycloalkyl (for example, 4-fluorocyclohexyl, 2-(trifluoromethyl)cyclopentyl, 3-N,N-dimethylcyclohexyl and the like), substituted alkoxy (for example methoxyethoxy, hydroxypropyloxy, methylenedioxy and the like), substituted aryl (for example, 4-hydroxyphenyl, 2,3-difluorophenyl, and the like), substituted aryloxy (for example, 4-hydroxyphenoxy, 2,3-difluorophenoxy, and the like), substituted arylalkyl (for example, 1-phenyl-ethyl, para-methoxyphenylethyl and the like), substituted heterocyclyl (for example, 5-chloro-pyridin-3-yl, 1-methyl-piperidin-4-yl and the like).

Unless specifically stated otherwise, where a compound may assume alternative stereoisomeric forms, all alternative isomers are intended to be encompassed within the scope of the present invention. It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

“Prodrug” is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

Modified release formulations as used herein refer to pharmaceutical formulations designed to release the drug or pharmaceutical in a manner that is different than an immediate release formulation. Thus, a modified release formulation may be a controlled release, sustained release, extended release, or delayed release formulation. Particular modified release formulations suitable for use in the invention are extended release formulations that release a drug or compound in the body over an extended period of time.

Unless specifically stated otherwise, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Unless specifically stated otherwise, the compounds of the present invention can exist in anhydrous as well as hydrated forms, such as the hexahydrate. In general, the hydrated forms are considered equivalent to the anhydrous forms for the purposes of the present invention.

The term “pharmaceutically acceptable salt” or “pharmaceutically acceptable equivalent” as used herein refers to a salt of an active compound of the compositions of the invention that possesses essentially the same pharmacological activity as the active compound and which is neither biologically nor otherwise undesirable. A pharmaceutically acceptable salt of a compound is one that, upon administration of a composition of the invention to a subject, is capable of providing the compound or an active metabolite or residue thereof. As known to those of skill in the art, “salts” of the compounds of the compositions of the invention may be derived from inorganic or organic acids and bases.

Examples of acids include, but are not limited to, 4-acetamidobenzoic acid, acetic acid, adipic acid, alginic acid, L-ascorbic acid, L-aspartic acid, benzenesulfonic acid, benzoic acid, butyric acid, (+)-camphoric acid, (+)-camphor-10-sulfonic acid, carbonic acid, cinnamic acid, citric acid, cyclamic acid, cyclopentanepropionic acid, decanoic acid, 2,2-dichloroacetic acid, dodecylsulfuric acid, ethane-1,2-disulfonic acid, ethanesulfonic acid, 2-hydroxy-ethanesulfonic acid, formic acid, fumaric acid, galactaric acid, gentisic acid, D-glucoheptonic acid, D-gluconic acid, D-glucuronic acid, glutamic acid, glutaric acid, 2-oxo-glutaric acid, glycerophosphoric acid, glycolic acid, heptanoic acid, hexanoic acid, hippuric acid, hydrobromic acid, hydrochloric acid, hydroiodic acid, 2-hydroxyethanesulfonic acid, isethionic, isobutyric acid, DL-lactic acid, lactobionic acid, lauric acid, maleic acid, (−)-L-malic acid, malonic acid, DL-mandelic acid, methanesulfonic acid, naphthalene-1,5-disulfonic acid, naphthalene-2-sulfonic acid, 1-hydroxy-2-naphthoic acid, nicotinic acid, nitric acid, octanoic acid, oleic acid, orotic acid, oxalic acid, palmitic acid, pamoic acid, pantothenic acid, pectinic acid, pectic acid, perchloric acid, phenylpropionic acid, phosphoric acid, picric acid, pivalic acid, propionic acid, (−)-L-pyroglutamic acid, salicylic acid, 4-amino-salicylic acid, sebacic acid, stearic acid, succinic acid, sulfuric acid, tartaric acid, (+)-L-tartaric acid, thiocyanic acid, toluenesulfonic acid, p-toluenesulfonic acid, and undecylenic acid. Examples of such bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like. For therapeutic use, salts of a compound of the compositions of the invention are contemplated as being pharmaceutically acceptable. However, salts of acids and bases that are non-pharmaceutically acceptable may also be used, for example, in the preparation or purification of a pharmaceutically acceptable compound.

Examples of pharmaceutically acceptable acids suitable for the preparation of pharmaceutically acceptable salts of an active compound include, but are not limited to, acetic acid, adipic acid, L-aspartic acid, benzenesulfonic acid, benzoic acid, citric acid, (+)-camphoric acid, (+)-camphor-10-sulfonic acid, dodecylsulfuric acid, D-gluconic acid, D-glucuronic acid, ethanesulfonic acid, fumaric acid, D-glucoheptonic acid, glycerophosphoric acid, hexanoic acid, hydrobromic acid, hydrochloric acid, 2-hydroxyethanesulfonic acid, isobutyric acid, DL-lactic acid, maleic acid, methanesulfonic acid, naphthalene-1,5-disulfonic acid, naphthalene-2-sulfonic acid, 1-hydroxy-2-naphthoic acid, nicotinic acid, oxalic acid, palmitic acid, propionic acid, succinic acid, sulfuric acid, (+)-L-tartaric acid, thiocyanic acid, p-toluenesulfonic acid, undecylenic acid, and the like. Other examples of salts include anions of an active compound combined with a suitable cation such as Na⁺, NH₄⁺, and NW₄⁺ (wherein W is a C₁₋₄ alkyl group), and the like.

The terms “effective amount” and “therapeutically effective amount” when used in reference to a composition of the invention refer to an amount or dosage sufficient to produce a desired therapeutic result. More specifically, a therapeutically effective amount is an amount of the composition sufficient to inhibit, for some period of time, one or more of the clinically defined pathological processes associated with the condition being treated. The effective amount may vary depending on the specific composition that is being used, and also depends on a variety of factors and conditions related to the patient being treated and the severity of the disorder. The determination of an effective amount or therapeutically effective amount of a given composition is well within the ability of those skilled in the art.

The term “methylphenidate” as used herein refers to a methylphenidate HCl, which is d,l (racemic)-threo-methyl α-phenyl-2-piperidineacetate hydrochloride (formula; C₁₄H₁₉NO₂.HCl), as well as pharmaceutically acceptable salts or equivalents thereof. In one method, use, or composition of the invention, the methylphenidate, or pharmaceutically acceptable equivalent thereof, is an extended release composition.

In another embodiment of the invention, the methylphenidate or equivalent thereof is replaced with a drug for treating attention deficit hyperactivity disorder (ADHD). Suitable drugs include, but are not limited to, Adderall® (amphetamine), Adderall XR® (amphetamine), Concerta® (methylphenidate hydrochloride), Daytrana® (methylphenidate), Desoxyn® (methamphetamine hydrochloride) Dexedrin® (dextroamphetamine sulfate), Focalin® (dexmethylphenidate hydrochloride), Focalin XR® (dexmethylphenidate hydrochloride), Metadate CD® (methylphenidate hydrochloride), Methylin® (methylphenidate hydrochloride), Ritalin® (methylphenidate hydrochloride), Ritalin SR® (methylphenidate hydrochloride), Ritalin LA® (methylphenidate hydrochloride), and Strattera® (atomoxetine hydrochloride).

Suitable compounds for use in the methods of the invention also include amfetamine transdermal, AZD5213 (histamine-3 receptor antagonist), bavisant (JNJ-31001074), COL-171, CX1739, EB-1020 (triple reuptake inhibitor), edivoxetine (LY2216684), KP106, KRL-401, methylphenidate extended-release (Purdue Pharma), NWP06 (methylphenidate extended-release suspension), NWP09 (methylphenidate extended-release chewable tablets), OPC-34712 (brexpiprazole), ORADUR-ADHD (sustained-release oral therapy), sofinicline, SPN-810 (molindone), SPN-811, SPN-812, TC-5619, and TD-9855.

In one embodiment of the invention, cognitive dysfunction, fatigue, or pain associated with fibromyalgia is treated or ameliorated by administering to a patient in need thereof, a therapeutically effective amount of a compound of Formula (I):

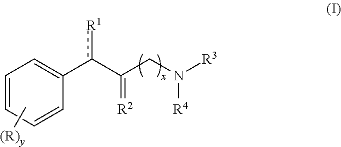

or stereoisomer, pharmaceutically acceptable salt, N-oxide, prodrug, hydrate, solvate or composition thereof,
wherein
R in each occurrence is independently selected from hydrogen, optionally substituted alkyl, optionally substituted alkoxy, acyl, halogen, —NR⁵R⁶ and hydroxy; or
two R groups on adjacent carbon atoms combine to form an aryl ring;
R⁵ and R⁶ are independently hydrogen, optionally substituted alkyl, optionally substituted arylalkyl or —C(O)OR⁷;
R¹ and R² are independently hydrogen, optionally substituted alkyl, optionally substituted alkoxy, optionally substituted aryl, optionally substituted arylalkyl, optionally substituted aryloxy, halogen, —NR⁵R⁶, hydroxy, oxo or —C(O)OR⁷; or
R¹ and R² combine through the carbon atoms to which they are bound to form an optionally substituted cycloalkyl ring;
R³ is independently hydrogen, optionally substituted alkyl, optionally substituted aryl, optionally substituted arylalkyl or —C(O)OR⁷; or
R² and R³ combine through the nitrogen atom to which R³ is bound to form an optionally substituted heterocycloalkyl ring;
R⁴ is hydrogen, optionally substituted alkyl, optionally substituted aryl or optionally substituted arylalkyl; or
R³ and R⁴ combine with the nitrogen atom to which they are bound to form a guanidinyl group;
R⁷ is hydrogen, optionally substituted alkyl, optionally substituted aryl, or optionally substituted arylalkyl;
y is 1 to 5; and
x is 0 to 2.

In another embodiment, R² and R³ combine through the nitrogen atom to which R³ is bound to form an optionally substituted piperidine ring, x is 0, and R¹ is —C(O)OR⁷.

In another embodiment R² and R³ combine through the nitrogen atom to which R³ is bound to form an optionally substituted piperidine ring, x is 0, R is hydrogen and R¹ is —C(O)OMe.

In another embodiment, the compound is methylphenidate (methyl 2-phenyl-2-(piperidin-2-yl)acetate), or a stereoisomer, pharmaceutically acceptable salt, N-oxide, prodrug, hydrate, solvate or composition thereof.

In another embodiment R² and R³ combine through the nitrogen atom to which R³ is bound to form an optionally substituted piperidine ring, x is 0, two R groups on adjacent carbon atoms combine to form an aryl ring and R¹ is —C(O)OMe.

In another embodiment, the compound is methylnaphthidate (methyl 2-(naphthalen-2-yl)-2-(piperidin-2-yl)acetate), or a stereoisomer, pharmaceutically acceptable salt, N-oxide, prodrug, hydrate, solvate or composition thereof.

In another embodiment, the compound is 4-methylmethylphenidate (methyl 2-(piperidin-2-yl)-2-p-tolylacetate), or a stereoisomer, pharmaceutically acceptable salt, N-oxide, prodrug, hydrate, solvate or composition thereof.

In one embodiment, R² and R³ combine through the nitrogen atom to which R³ is bound to form an optionally substituted piperidine ring, x is 0, R is halogen and R¹ is —C(O)OMe.

In another embodiment, the compound is 3-chloromethylphenidate (methyl 2-(3-chlorophenyl)-2-(piperidin-2-yl)acetate), or a stereoisomer, pharmaceutically acceptable salt, N-oxide, prodrug, hydrate, solvate or composition thereof.

In another embodiment, the compound is 4-fluoromethylphenidate (methyl 2-(4-fluorophenyl)-2-(piperidin-2-yl)acetate), or a stereoisomer, pharmaceutically acceptable salt, N-oxide, prodrug, hydrate, solvate or composition thereof.

In another embodiment, the compound is 3,4-dichloromethylphenidate (methyl 2-(3,4-dichlorophenyl)-2-(piperidin-2-yl)acetate), or a stereoisomer, pharmaceutically acceptable salt, N-oxide, prodrug, hydrate, solvate or composition thereof.

In another embodiment, R¹ is hydrogen and R² and R³ are optionally substituted alkyl.

In another embodiment, R¹ is hydrogen and R, R² and R³ are optionally substituted alkyl.

In another embodiment, R is halogen, R¹ is hydrogen, and R² and R³ are optionally substituted alkyl.

In another embodiment, R and R¹ are hydrogen and R² and R³ are optionally substituted alkyl.

In another embodiment, R and R¹ are hydrogen, R² and R³ are optionally substituted alkyl, and x is 0.

In another embodiment, the compound is amphetamine (N-methyl-2-phenylethanamine), or a stereoisomer, pharmaceutically acceptable salt, N-oxide, prodrug, hydrate, solvate or composition thereof.

In another embodiment, the compound is methamphetamine (N-methyl-1-phenylpropan-2-amine), or a stereoisomer, pharmaceutically acceptable salt, N-oxide, prodrug, hydrate, solvate or composition thereof.

In another embodiment, R is halogen, R¹ is oxo, and R² and R³ are optionally substituted alkyl.

In another embodiment, the compound is bupropion (2-(tert-butylamino)-1-(3-chlorophenyl)propan-1-one), or a stereoisomer, pharmaceutically acceptable salt, N-oxide, prodrug, hydrate, solvate or composition thereof.

In another embodiment, R¹ and R² combine through the carbon atoms to which they are bound to form an optionally substituted cycloalkyl ring, R² and R³ are optionally substituted alkyl, and x is 0.

In another embodiment, R¹ and R² combine through the carbon atoms to which they are bound to form an optionally substituted bicyclo[2.2.1]heptane ring, R is hydrogen, R² and R³ are optionally substituted alkyl, and x is 0.

In another embodiment, the compound is fencamfamine (N-ethyl-3-phenylbicyclo[2.2.1]heptan-2-amineine), or a stereoisomer, pharmaceutically acceptable salt, N-oxide, prodrug, hydrate, solvate or composition thereof.

In another embodiment, R¹ is optionally substituted phenoxy and R² and R³ are optionally substituted alkyl.

In another embodiment, R is halogen or optionally substituted alkyl, R¹ is optionally substituted phenoxy and R² and R³ are optionally substituted alkyl.

In another embodiment, R is hydrogen, R¹ is optionally substituted phenoxy and R² and R³ are optionally substituted alkyl.

In another embodiment, R is hydrogen, R¹ is 2-methylphenoxy and R² and R³ are optionally substituted alkyl.

In another embodiment, the compound is atomoxetine (N,2-dimethyl-3-phenyl-3-(o-tolyloxy)propan-1-amine), or a stereoisomer, pharmaceutically acceptable salt, N-oxide, prodrug, hydrate, solvate or composition thereof.

In another embodiment, R³ and R⁴ combine with the nitrogen atom to which they are bonded to form a guanidinyl group.

In another embodiment, R is halogen and R³ and R⁴ combine with the nitrogen atom to which they are bonded to form a guanidinyl group.

In another embodiment, R is halogen, R² is oxo and R³ and R⁴ combine with the nitrogen atom to which they are bonded to form a guanidinyl group.

In another embodiment, the compound is guanfacine (N-(diaminomethylene)-2-(2,6-dichlorophenyl)acetamide), or a stereoisomer, pharmaceutically acceptable salt, N-oxide, prodrug, hydrate, solvate or composition thereof.

In another embodiment of the invention, cognitive dysfunction, fatigue, or pain associated with fibromyalgia is treated or ameliorated by administering to a patient in need thereof, a therapeutically effective amount of a composition comprising one or more compounds of Formula I or a stereoisomer, pharmaceutically acceptable salt, N-oxide, prodrug, hydrate or solvate thereof.

In one embodiment of the invention, cognitive dysfunction, fatigue, or pain associated with fibromyalgia is treated or ameliorated by administering to a patient in need thereof, a therapeutically effective amount of a composition comprising one or more of risperidone (4-[2-[4-(6-fluorobenzo[d]isoxazol-3-yl)-1-piperidyl]ethyl]-3-methyl-2,6-diazabicyclo[4.4.0]deca-1,3-dien-5-one), modafinil (2-(benzhydrylsulfinyl)acetamide), bavisant ((4-cyclopropylpiperazin-1-yl)[4-(morpholin-4-ylmethyl)phenyl]methanone), edivoxetine ((1R)-2-(5-fluoro-2-methoxyphenyl)-1-[(2S)-morpholin-2-yl]-1-(tetrahydro-2H-pyran-4-yl)ethanol), brexpiperazole (7-{4-[4-(1-benzothiophen-4-yl)piperazin-1-yl]butoxy}quinolin-2(1H)-one), sofinicline (3-(5,6-Dichloro-pyridin-3-yl)-1S,5 S-3,6-diazabicyclo[3.2.0]heptane) or a stereoisomer, pharmaceutically acceptable salt, N-oxide, prodrug, hydrate or solvate thereof.

In another embodiment of the invention, cognitive dysfunction, fatigue, or pain associated with fibromyalgia is treated or ameliorated by administering to a patient in need thereof, a therapeutically effective amount of a composition comprising one or more compounds of Formula I, or a stereoisomer, pharmaceutically acceptable salt, N-oxide, prodrug, hydrate or solvate thereof, and optionally comprising one or more of risperidone (4-[2-[4-(6-fluorobenzo[d]isoxazol-3-yl)-1-piperidyl]ethyl]-3-methyl-2,6-diazabicyclo[4.4.0]deca-1,3-dien-5-one), modafinil (2-(benzhydrylsulfinyl)acetamide), bavisant ((4-cyclopropylpiperazin-1-yl)[4-(morpholin-4-ylmethyl)phenyl]methanone), edivoxetine ((1R)-2-(5-fluoro-2-methoxyphenyl)-1-[(2S)-morpholin-2-yl]-1-(tetrahydro-2H-pyran-4-yl)ethanol), brexpiperazole (7-{4-[4-(1-benzothiophen-4-yl)piperazin-1-yl]butoxy}quinolin-2(1H)-one), sofinicline (3-(5,6-Dichloro-pyridin-3-yl)-1S,5 S-3,6-diazabicyclo[3.2.0]heptane) or a stereoisomer, pharmaceutically acceptable salt, N-oxide, prodrug, hydrate or solvate thereof.

In another embodiment, the composition comprising one or more compounds of Formula I, or a stereoisomer, pharmaceutically acceptable salt, N-oxide, prodrug, hydrate or solvate thereof, and optionally comprising one or more of risperidone (4-[2-[4-(6-fluorobenzo[d]isoxazol-3-yl)-1-piperidyl]ethyl]-3-methyl-2,6-diazabicyclo[4.4.0]deca-1,3-dien-5-one), modafinil (2-(benzhydrylsulfinyl)acetamide), bavisant ((4-cyclopropylpiperazin-1-yl)[4-(morpholin-4-ylmethyl)phenyl]methanone), edivoxetine ((1R)-2-(5-fluoro-2-methoxyphenyl)-1-[(2S)-morpholin-2-yl]-1-(tetrahydro-2H-pyran-4-yl)ethanol), brexpiperazole (7-{4-[4-(1-benzothiophen-4-yl)piperazin-1-yl]butoxy}quinolin-2(1H)-one), sofinicline (3-(5,6-Dichloro-pyridin-3-yl)-1S,5S-3,6-diazabicyclo[3.2.0]heptane) or a stereoisomer, pharmaceutically acceptable salt, N-oxide, prodrug, hydrate or solvate thereof, is of a modified release formulation.

### 2. EXAMPLES

The Examples that follow are illustrative of specific embodiments of the invention, and various uses thereof. They are set forth for explanatory purposes only, and should not be construed as limiting the scope of the invention in any way.

### Example 1

### Development of the Mental Clutter Scale

Memory loss in fibromyalgia is often characterized by a distinctive combination of disturbances that sets people with fibromyalgia (FMS) apart from other medical patients with memory complaints. The view has arisen, that both forgetfulness and mental fog characterize their condition, suggesting that memory and mental clarity deteriorate together in FMS (Leavitt et al., 2002, “Cognitive and dissociative manifestations in fibromyalgia,” J. Clin. Rheumatol. 8(2): 77-84). By contrast, deterioration in memory is not accompanied by disturbances in mental clarity in other patients reporting memory loss. The great majority of cognitively-compromised patients reported memory loss, but good mental clarity, with only 8.8% of a sample connecting memory disturbance to diminished mental clarity.

The label “fibrofog” entered the clinical language as a convenient term to describe the symptoms of a combination of memory loss and mental fog. The pattern of cognitive dysfunction in FMS may be different from other patients with cognitive problems. To investigate this cognitive model, a new scale was developed to capture the varying appreciation of disturbances, especially the combination of memory complaints and the lack of mental clarity, in the cognitive state of patients with fibromyalgia.

Three studies involving a sample of over 800 subjects were carried out to determine the structure and stability of a new scale, the Mental Clutter Scale (MCS) (Leavitt & Katz, 2011, “Development of the Mental Clutter Scale,” Psychol. Rep. 109(2): 445-52). An initial pool of 13 items was formulated from self-statements of patients presenting with a history of memory complaints and a review of the literature. Seven items relating to problems with cognitive skills were rated on a 10-point Likert scale from 1=no problem, to 10=severe problem. Six items relating to the frequency of diminished mental clarity were rated on a 10-point Likert scale from 1=not at all, to 10=all the time.

The 13-item measure was administered to a sample of 223 patients with various rheumatic disorders. Data of 88 patients who met the ACR criteria for FMS were subjected to factor analysis with varimax rotation. The criteria of Eigenvalue greater than one, combined with a visual inspection of the Scree Plot were used in identifying the number of factors to be extracted. A 2-factor structure solution met these criteria.

Studies 2 and 3 were used to confirm factor structure found in the initial sample. For studies 2 and 3, the scales were modified by adding one additional item to Factor 1 (mental speed) and two additional items to Factor 2 (fuzzy headedness and information overload).

Sample 1 comprised 88 females with memory complaints who met ACR criteria for fibromyalgia. They had a mean age of 50.3±10.5 years and a mean level of education of 15.0±3.7 years. Sample 2 comprised of 128 FMS subjects (120 females and 8 males) with memory complaints drawn from the same clinical setting. They had a mean age of 49.5±11.7 years and a mean level of education of 14.3±2.0 years. Sample 3 consisted of 592 subjects (523 females, and 69 males) with memory complaints that completed a web-based version of the scale over the Internet. The median age of the sample was 48 years.

Study 1

The factor analysis produced a two-factor solution (Eigenvalues=9.6 and 1.1 respectively) with 7 variables loading highly (≧0.7) on the first factor and six variables loading highly on the second factor (Table 1). Factor loadings of (≧0.7) indicate a close association of variables with a factor and formed the basis for inclusion of variables in Factors. Seven variables cover a broad range of cognitive skills and formed the Cognition Factor (I). They are shown in bold in Table 1. Six variables associated mainly with intrinsic qualities of the brain relating to clear headedness formed the Mental Clarity Factor (II). The two factors explained 82.4% of the total variance.

TABLE 1
Comparison of Factor Loadings Across Three Studies

| | | | Study 2 | | Study 3 | |
|---|---|---|---|---|---|---|
| | Study 1 | | Replication | | Replication | |
| | Factor 1 | Factor 2 | Factor 1 | Factor 2 | Factor 1 | Factor 2 |
| Concentration | .87 | .37 | .82 | .36 | .79 | .36 |
| Memory | .75 | .47 | .83 | .28 | .75 | .31 |
| Staying | .87 | .32 | .85 | .28 | .78 | .36 |
| Focused | | | | | | |
| Multitasking | .81 | .38 | .77 | .41 | .75 | .26 |
| Expressing Self | .83 | .38 | .74 | .36 | .66 | .34 |
| Thinking | .86 | .44 | .82 | .40 | .78 | .44 |
| Clearly | | | | | | |
| Perceptual | .78 | .48 | .80 | .40 | .77 | .39 |
| Clarity | | | | | | |
| Mental Speed | | | .81 | .39 | .78 | .33 |
| Spaciness | .49 | .71 | .55 | .62 | .43 | .69 |
| Haziness | .29 | .91 | .34 | .83 | .42 | .74 |
| Confusion | .49 | .73 | .50 | .73 | .49 | .72 |
| Cluttered | .42 | .82 | .40 | .81 | .44 | .77 |
| Thinking | | | | | | |
| Fogginess | .41 | .85 | .44 | .82 | .56 | .69 |
| Rushing | .32 | .75 | .24 | .78 | .12 | .77 |
| Thoughts | | | | | | |
| Fuzzy | | | .36 | .84 | .40 | .75 |
| headedness | | | | | | |
| Information | | | .30 | .80 | .37 | .70 |
| overload | | | | | | |

Studies 2 and 3

Confirmatory factor analysis in studies 2 and 3 produced highly similar 2 factor solutions, with Eigenvalues of 11.0 and 1.5 in study 2 accounting for 77.7% of the variance, and 10.1 and 1.2 in study 3 and accounting for 70.7% of the variance. The two-factor solution across studies 2 and 3 displays a high degree of overlap with the original 2 factor inventory of variables indicating a high amount of factor stability.

Group differences were examined in the presentation of cognitive difficulties on the basis of this two dimensional framework. As can be seen in Table 2, groups with and without memory complaints differed on both factor scales. Those with memory complaints on average produced significantly higher scores on both cognitive skills and mental clutter. Higher scores represent increased disturbance in cognitive skills and mental clarity. Individuals with fibromyalgia presented the highest level of disturbance in both cognitive skills and mental clarity.

TABLE 2
Group Differences in the Presentation of Cognitive
Difficulties on the Basis of a Two Dimensional Framework
Involving Cognitive Skills and Mental Clutter

| | Cognitive | Mental Clutter |
|---|---|---|
| Fibromyalgia Sample | | |
| Memory complaints (142) | 5.7(2.0)ₐ ***^{#} | 5.0(2.1) ***^{#} |
| No memory complaints (56) | 2.2(1.3) | 2.0(1.4) |
| Medical Sample (n = 207) | | |
| Memory complaints (n = 80) | 4.4(2.2) *** | 3.2(2.1) *** |
| No memory complaints (n = 207) | 1.6(0.9) | 1.4(0.8) |
| Community Sample | | |
| Memory complaints (n = 47) | 3.1(1.8) *** | 2.4(1.5) *** |
| No memory complaints (n = 116) | 1.4(0.8) | 1.4(0.8) |

ₐFrequency ratings: 1 Not at all to 10 All the Time.
*** p < 0.001 memory vs. no-memory complaints
^{#}p < 0.05 Fibromyaliga vs. Medical Sample Comparison
## p < 0.01 Fibromyaliga vs. Medical Sample Comparison

### Example 2

### Improved Cognitive Function with Methylphenidate

Abnormalities in naming speed are an unappreciated feature of cognitive dysfunction in fibromyalgia (FMS). Approximately 50% of FMS patients with memory problems name words at a rate that is 203 milliseconds slower than normal. The connection between naming speed and memory loss in FMS is unclear. Stimulant medications like methylphenidate have been known to influence naming speed and could provide clues to the relationship between cognitive functioning and naming speed. The purpose of this experiment was to determine if faster naming speed is connected to positive change in cognitive functioning in patients with fibromyalgia.

A word naming speed measure (Stroop Color and Word Test) and a measure of cognitive functioning (Mental Clutter Scale, MCS) were administered to 15 patients with FMS, before receiving methylphenidate and post-treatment with methylphenidate. The FMS patients were female, met American College of Rheumatology criteria for FMS, and had memory problems. Methylphenidate dosage was clinically determined and ranged from 10 to 30 mg per day. The median time of methylphenidate usage at re-testing was 30 days. Naming speed was determined by the number of words named in a 45 second time period.

The mean age of the FMS sample was 46.3±11.6 years with 14.1±2.1 years of education. Twelve of 15 FMS patients showed a significant reduction in time needed to name words post-methylphenidate treatment. Pre-methylphenidate, the patients read 77.4 words in 45 seconds (581.4 msec/word). Post-methylphenidate, the patients read 93 words in 45 seconds (483.9 msec/word). This represents a 97.5 millisecond benefit from methylphenidate. The normative sample reads 108 words in 45 seconds or 417 msec/word. Post-methylphenidate changes on the Cognition and Mental Clarity subscales of the MCS are shown in Table 3. Scale items for the MCS are displayed in Table 4. FMS patients showed a 17 point improvement with methylphenidate on Cognition, and a 19 point improvement on Mental Clarity.

TABLE 3
Pre-Methylphenidate and Post-Methylphenidate Scores on the Cognition
and Mental Clarity Subscales of the Mental Clutter Scale

| | Pre-methylphenidate | Post-methylphenidate ^{a} |
|---|---|---|
| Cognition | 52.8 ± 15.6 | 35.1 ± 11.3** |
| Mental Clarity | 48.1 ± 16.5 | 29.2 ± 13.6** |

^{a} Lower scores represent improved performance,
**p < 0.01

TABLE 4
Items of the Cognition and Mental Clarity
Subscales of the Mental Clutter Scale

| Cognition Subscale | Mental Clarity Subscale |
|---|---|
| Concentration | Spaciness |
| Memory | Haziness |
| Staying Focused | Confusion |
| Multitasking | Cluttered Thinking |
| Expressing Self | Fogginess |
| Thinking Clearly | Rushing Thoughts |
| Perceptual Clarity | Fuzzy headedness |
| Mental Speed | Information Overload |

Methylphenidate appears to have short term benefits for naming speed and cognitive functioning in fibromyalgia. It quickly allows patients with FMS to operate at a more normal pace in naming words and broadly improves cognitive functioning. Elimination of the 97.5 millisecond time lag is thought to reset the neural clock in FMS so that word information is back in-sync with other streams of neural information. The benefits of methylphenidate clearly show a connection between faster naming speed and improved cognition in patients with fibromyalgia. Surprisingly, the inventors have seen that many patients report significant improvement in all their symptoms, including cognitive dysfunction, fatigue, and pain, with the use of methylphenidate.

### Example 3

### Treatment of Fibromyalgia with Atomoxetine HCl

Strattera™ (atomoxetine HCl) represents an important alternative to the family of methylphenidate medications in the treatment of symptoms associated with fibromyalgia. While the mode of therapeutic action has yet to be established, there is a general presumption that most attention regulating medications act as gatekeepers in the brain, helping the brain to better regulate the flow of information. Atomoxetine HCl is presumed to work by blocking the reuptake of norepinephrine. As such, it seems reasonable to assume that beneficial effects achieved with methylphenidate in the previous study should also be achievable with atomoxetine HCl.

Eleven fibromyalgia patients with unexplained memory problems of at least six months duration were treated with atomoxetine HCl for 4 weeks. A Memory Functioning Questionnaire was used to assess the effects of atomoxetine on the cognitive response of adult patients. Six of 11 fibromyalgia patients displayed at least a 15-point improvement, as assessed by the Memory Functioning Questionnaire, following treatment with atomoxetine HCl. This indicates that atomoxetine HCl was able to demonstrate a positive effect on cognitive function as early as four weeks.

### Example 4

### Methylphenidate Improves Concentration, Energy, and Mood in Fibromyalgia

Fibromyalgia patients were treated with methylphenidate and the effects on concentration, energy, and feelings of well-being were measured. Interference from pain on energy, mood, and concentration was measured on a 10 point visual analogue scale, with the endpoint of 1 indicating that pain does not interfere, and the endpoint of 10 indicating that pain completely interferes. Pain intensity was also recorded on a 10 point visual analogue scale, with the endpoint of 1 indicating no pain, and the endpoint of 10 indicating severe pain. The four measures were administered to 48 patients with FMS, before receiving methylphenidate and post methylphenidate treatment. The FMS patients were female and met the new ACR criteria for FMS. Methylphenidate dosage was clinically determined and ranged from 10 to 60 mg per day. The median methylphenidate usage at re-testing was 30 days.

The mean age of the FMS sample was 48.3±12.3 years. Table 5 shows test performance at the pretest and post-test sessions. A repeated measures ANOVA showed significant reduction in pain interference on energy (p<0.01), concentration (p<0.001), and mood (p<0.05). The largest change occurred with concentration. However, a significant change in pain intensity with methylphenidate treatment was not observed.

TABLE 5
Pre-methylphenidate and Post-methylphenidate Scores
on Concentration, Energy, Mood, and Pain

| | Pre-methylphenidate | Post-methylphenidate ^{a} |
|---|---|---|
| Concentration | 6.5 ± 2.3 | 5.4 ± 2.4** |
| Energy | 6.6 ± 2.4 | 5.9 ± 2.2*** |
| Mood | 6.0 ± 2.6 | 5.3 ± 2.5* |
| Pain | 6.7 ± 1.9 | 6.4 ± 2.4 |

*p < 0.05
**p < 0.01
***p < 0.001
^{a} = Lower scores represent improved performance

Although a significant change in pain intensity was not observed in this analysis, methylphenidate was found to be helpful in alleviating some of the problems patients with fibromyalgia have with concentration, energy, and mood. Surprisingly, as a result of methylphenidate treatment, patients with fibromyalgia report functional improvement, but no change in pain intensity. This paradox may be attributed to the phenomenon of sensory adaptation. To illustrate sensory adaptation, think about visual reaction to extremely bright sunlight after emerging from a dark room. Initially, a person may be blinded, but in matter of seconds the receptors of the eye adjust and they see normally even though the intensity of the sunlight has not changed. Methylphenidate could in a similar way operate to facilitate the adaptation to pain in fibromyalgia without changing the level of pain intensity. Thus, sensory adaptation may explain why the inventors have seen a number of fibromyalgia patients who, when treated with methylphenidate, reported that they felt better, including reduced pain. This study provides surprising results to support the benefits of methylphenidate on energy, concentration, mood, and overall functional improvement in patients with fibromyalgia, and despite finding a lack of significant change on pain intensity scored here, patients reported the sensation of decreased pain.

While the invention has been described in terms of various embodiments, it is understood that variations and modifications will occur to those skilled in the art. Therefore, it is intended that the appended claims cover all such equivalent variations that come within the scope of the invention as claimed. In addition, the section headings used herein are for organizational purposes only and are not to be construed as limiting the subject matter described.

All references cited in this application are expressly incorporated by reference herein.

What is claimed is:

1. A method for treating or ameliorating cognitive dysfunction, fatigue, energy, concentration, mood, or pain associated with fibromyalgia comprising administering to a patient in need thereof a therapeutically effective amount of a composition comprising a compound of Formula (I): 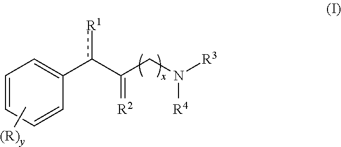
or a stereoisomer, pharmaceutically acceptable salt, N-oxide, prodrug, hydrate, or solvate thereof,
wherein
R in each occurrence is independently selected from hydrogen, optionally substituted alkyl, optionally substituted alkoxy, acyl, halogen, —NR⁵R⁶ and hydroxy; or
two R groups on adjacent carbon atoms combine to form an aryl ring;
R⁵ and R⁶ are independently hydrogen, optionally substituted alkyl, optionally substituted arylalkyl or —C(O)OR⁷;
R¹ and R² are independently hydrogen, optionally substituted alkyl, optionally substituted alkoxy, optionally substituted aryl, optionally substituted arylalkyl, optionally substituted aryloxy, halogen, —NR⁵R⁶, hydroxy, oxo or —C(O)OR⁷; or
R¹ and R² combine through the carbon atoms to which they are bound to form an optionally substituted cycloalkyl ring;
R³ is independently hydrogen, optionally substituted alkyl, optionally substituted aryl, optionally substituted arylalkyl or —C(O)OR⁷; or
R² and R³ combine through the nitrogen atom to which R³ is bound to form an optionally substituted heterocycloalkyl ring;
R⁴ is hydrogen, optionally substituted alkyl, optionally substituted aryl or optionally substituted arylalkyl; or
R³ and R⁴ combine with the nitrogen atom to which they are bound to form a guanidinyl group;
R⁷ is hydrogen, optionally substituted alkyl, optionally substituted aryl, or optionally substituted arylalkyl;
y is 1 to 5; and
x is 0 to 2.

2. The method of claim 1, wherein the compound is methylphenidate (methyl 2-phenyl-2-(piperidin-2-yl)acetate), methylnaphthidate (methyl 2-(naphthalen-2-yl)-2-(piperidin-2-yl)acetate), 4-methylmethylphenidate (methyl 2-(piperidin-2-yl)-2-p-tolylacetate), 3-chloromethylphenidate (methyl 2-(3-chlorophenyl)-2-(piperidin-2-yl)acetate), 4-fluoromethylphenidate (methyl 2-(4-fluorophenyl)-2-(piperidin-2-yl)acetate), 3,4-dichloromethylphenidate (methyl 2-(3,4-dichlorophenyl)-2-(piperidin-2-yl)acetate), amphetamine (N-methyl-2-phenylethanamine), methamphetamine (N-methyl-1-phenylpropan-2-amine), bupropion (2-(tert-butylamino)-1-(3-chlorophenyl)propan-1-one), fencamfamine (N-ethyl-3-phenylbicyclo[2.2.1]heptan-2-amineine), atomoxetine (N,2-dimethyl-3-phenyl-3-(o-tolyloxy)propan-1-amine) or guanfacine (N-(diaminomethylene)-2-(2,6-dichlorophenyl)acetamide).

3. The method of claim 2, wherein the compound is methylphenidate (methyl 2-phenyl-2-(piperidin-2-yl)acetate)

4. The method of claim 1, wherein the composition is administered in a tablet dosage form.

5. The method of claim 4, wherein the tablet dosage form comprises an amount of methylphenidate, or a pharmaceutically acceptable equivalent thereof, wherein the amount is selected from the group consisting of 1 mg, 5 mg, 10 mg, 15 mg, 20 mg, 25 mg, 30 mg, 35 mg, 40 mg, 45 mg, 50 mg, 55 mg, 60 mg 65 mg and 70 mg.

6. The method of claim 1, wherein the composition is administered in a tablet dosage form two or three times daily.

7. The method of claim 1, wherein the composition is administered at least once a day for at least four weeks.

8. The method of claim 1, wherein composition is of a modified release formulation.

9. The method of claim 1, wherein the treatment results in a reduction in cognitive dysfunction associated with fibromyalgia.

10. The method of claim 1, wherein the treatment results in a reduction in fatigue associated with fibromyalgia.

11. The method of claim 1, wherein the treatment results in a reduction in pain associated with fibromyalgia.

12. The method of claim 1, wherein the treatment results in improved concentration in the patient.

13. The method of claim 1, wherein the treatment results in improved mood in the patient.

14. The method of claim 1, wherein the treatment results in improved energy in the patient.

15. The method of claim 1, wherein the treatment results in functional improvement in the patient.

16. A method for treating or ameliorating cognitive dysfunction, fatigue, energy, concentration, mood, or pain associated with fibromyalgia comprising administering to a patient in need thereof, a therapeutically effective amount of a composition comprising one or more compounds of Formula I, or a stereoisomer, pharmaceutically acceptable salt, N-oxide, prodrug, hydrate or solvate thereof, and optionally comprising one or more of risperidone (4-[2-[4-(6-fluorobenzo[d]isoxazol-3-yl)-1-piperidyl]ethyl]-3-methyl-2,6-diazabicyclo[4.4.0]deca-1,3-dien-5-one), modafinil (2-(benzhydrylsulfinyl)acetamide), bavisant ((4-cyclopropylpiperazin-1-yl)[4-(morpholin-4-ylmethyl)phenyl]methanone), edivoxetine ((1R)-2-(5-fluoro-2-methoxyphenyl)-1-[(2S)-morpholin-2-yl]-1-(tetrahydro-2H-pyran-4-yl)ethanol), brexpiperazole (7-{4-[4-(1-benzothiophen-4-yl)piperazin-1-yl]butoxy}quinolin-2(1H)-one), sofinicline (3-(5,6-Dichloro-pyridin-3-yl)-1S,5S-3,6-diazabicyclo[3.2.0]heptane) or a stereoisomer, pharmaceutically acceptable salt, N-oxide, prodrug, hydrate or solvate thereof.
